(19) United States
(12) Patent Application Publication
(10) Pub. No.: US 2008/0026470 A1
(43) Pub. Date: Jan. 31, 2008

(54) Device and Method for Determining the Content of Phenolic Components in Must and Wine

(75) Inventors: Dieter Tanzer, Rossdorf (DE); Uwe Kraetschmer, Breuberg (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, Darmstadt (DE)

(21) Appl. No.: 11/573,846

(22) PCT Filed: Jul. 30, 2005
(86) PCT No.: PCT/EP05/08280

§ 371 Date: Feb. 16, 2007

(30) Foreign Application Priority Data
Aug. 20, 2004 (DE) 10 2004 040 343.0

(51) Int. Cl. G01N 33/14; G01N 31/22

(52) U.S. Cl. 436/24

(57) ABSTRACT

The invention relates to a device which is in the form of a test strip, and to a method for quickly determining the content of polyphenoles in must and wine. Detection occurs after the method for folin and ciocalteau by means of a test strip which is impregnated with the reagent, which is produced in a method wherein at least sodium, sodium molybdate and phosphoric acid is mixed and heated.

The invention relates to a means in the form of a test strip and a method for the rapid determination of the content of polyphenols in must and wine.

Must and wine comprise a multiplicity of phenolic compounds which essentially belong to 4 substance groups and are grouped together under the name polyphenols or total polyphenols: phenolcarboxylic acids, flavones, anthocyans and catechins (tannins). Precise knowledge of these compounds and the properties thereof is still relatively new since only chromatographic methods facilitated differentiation of the components, which are difficult to separate. They have a crucial influence on the flavour and aroma of a wine. On the one hand, in particular in white wines, they can result in a bitter taste, on the other hand, in particular in red wines, they can shape the variety-typical structure of the wines and favour maturing and the development of body. In red wines, they additionally have a crucial influence on the development of the colour intensity. Wines classified as being deficient in flavour frequently have an excessively high polyphenol content. Such wines are referred to, for example, as grassy, tannic, dry, woody or simply as bitter. Red wines having an excessively high polyphenol content are rough and hard instead of smooth.

The polyphenol content can be influenced by specific control of the grape processing (for example type of grape pressing, must standing time, must heating, etc.). Taste deficiencies caused by polyphenols present in excess can be eliminated or at least reduced by aftertreatment with suitable fining agents, such as, for example, gelatine, polyvinylpolypyrrolidone or activated carbon.

The prerequisite for taking suitable measures during winemaking is knowledge of the total polyphenol content, which is in the region of about 200 mg/l in the case of white wines and a factor of about 10 higher in the case of red wines (Würdig, Wohler: Chemie des Weines, Handbuch der Lebensmitteltechnologie [Chemistry of Wine, Handbook of Food Technology] (1989), Verlag Eugen Ulmer, Stuttgart, for example pp. 571 ff). For determination of the total polyphenols, the Folin-Ciocalteau method is recognised (Bergner, Lemperle: Weinkompendium [Wine Compendium] (1998) Verlag Hirzel, Stuttgart, p. 222). With the Folin-Ciocalteau reagent, a mixture of phosphotungstic and phosphomolybdic acid, in alkaline medium, phenolic substances give a blue coloration, which can be evaluated with the aid of a suitable spectrophotometer.

Owing to the necessity for a spectrophotometer and the reagents necessary for the reaction, as well as the complexity of the evaluation, the use of the determination method is the preserve of corresponding laboratories with suitable skilled personnel. A simple determination method for rapid decision-making directly in the winery has to date not been available to a vintner, for which reason he has to rely exclusively on his sensory judgement ability during winemaking.

The present invention is therefore based on the object of providing a simple method for the determination of the total polyphenol content in must/wine samples which can be used directly in the winery. The method must be quick to carry out, inexpensive and readily storable in the form of a ready-to-use test kit. In particular, the method according to the invention should be open not only to semiquantitative, visual evaluation, but also to quantitative evaluation using a reflectometer.

It has been found that the reagents used in the wet-chemical Folin-Ciocalteau method can also be applied to a test strip under certain conditions and thus dry-chemical determination of the polyphenol content can be made available.

The invention therefore relates to a test strip for the determination of the total content of phenolic components in must and wine, characterised in that the test strip is impregnated with a reagent obtained by mixing and heating at least sodium tungstate, sodium molybdate and o-phosphoric acid.

In a preferred embodiment, the reagent with which the test strip is impregnated additionally comprises lithium sulfate.

In another preferred embodiment, the reagent with which the test strip is impregnated additionally comprises hydrochloric acid.

In another preferred embodiment, the reagent with which the test strip is impregnated is prepared using sodium tungstate in concentrations between 5 and 50 g/l, sodium molybdate in concentrations between 1 and 25 g/l and lithium sulfate in concentrations between 5 and 50 g/l.

In a particularly preferred embodiment, the test strip is impregnated with a reagent which has been prepared by
a) mixing sodium tungstate and sodium molybdate in water
b) adding o-phosphoric acid and hydrochloric acid
c) boiling the mixture under reflux for a number of hours
d) adding lithium sulfate.

The present invention also relates to a method for the determination of the total content of phenolic components in must and wine, characterised by the following method steps:
a) provision of an aqueous sample and a test strip according to the invention
b) alkalisation of the sample by addition of a base
c) brief dipping of the test strip into the sample
d) visual and/or spectroscopic evaluation of the test strip.

In a preferred embodiment, the alkalisation in step b) is carried out using concentrated sodium hydroxide solution.

The present invention also relates to a kit for the determination of the total content of phenolic components in must and wine, at least comprising one or more test strips according to the invention and a base for alkalisation of the sample.

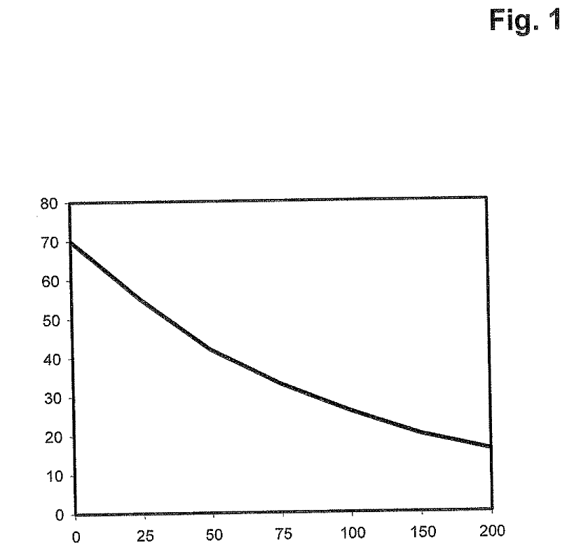

FIG. 1 shows a calibration curve for the reflectometric determination of polyphenols. Further details are given in Example 1.

In accordance with the invention, must and wine denotes any liquid produced from grapes as well as other liquids, such as fruit juices, beer or other alcoholic beverages, but preferably grape juice, grape must or wine.

In accordance with the invention, an aqueous sample solution is a solution which comprises must and wine in undiluted or diluted form. In the case of wines, for example, the aqueous sample solution often comprises the wine diluted 1:5 to 1:10 with water since the determination then gives better results.

Phenolic components or polyphenols are all substances occurring in must and wine which carry at least one phenolic radical. In particular, these are phenoicarboxylic acids, flavones, anthocyans and catechins (tannins).

The structure of a test strip is known to the person skilled in the art. It typically consists of a solid material, for example plastic or stable paper, to at least part of which a reaction zone comprising a sorptive or swellable support material has been applied. The support material of the reaction zone is impregnated with the reagents necessary for the respective determination, i.e. it is wetted with the corresponding reagents and subsequently dried.

The phenolic components are determined by means of the test strip according to the invention based on the Folin and Ciocalteau method (V. L. Singleton et al., Methods in Enzymology, 1999, 299, 152-178).

In contrast to all Folin and Ciocalteau determination systems known to date, the determination system according to the invention is in the form of an impregnated matrix, i.e. all reagents necessary for the determination of phenolic components (colouring reagent and optionally stabilisers and solubilisers) are embedded in a sorptive or swellable support. The determination reaction proceeds after contact of the reaction zone with the sample. The colour formed is a measure of the amount of analyte to be determined and is evaluated reflectometrically or visually by comparison with a colour card.

The sorptive supports used can be any materials which are usually used for such tests. The most widespread is the use of filter paper, but it is also possible to employ other sorptive cellulose or plastic products. However, it should be noted that reducing substances present in the support materials already react with the determination reagents and can result in a blue coloration of the test strip. The support material should therefore contain no or only small amounts of reducing substances in order that the blank value does not become too high.

The sorptive supports are impregnated in a known manner with impregnation solutions which comprise all reagents necessary for the determination. The impregnation must be carried out very substantially with exclusion of light since the reagent system is light-sensitive. The impregnated and dried papers can be cut to suitable size and adhesively bonded or heat-sealed onto support films in a known manner.

The impregnation solutions according to the invention are obtained by preparing and heating an aqueous solution of at least sodium tungstate, sodium molybdate and phosphoric acid. Lithium sulfate is preferably additionally added after the heating.

The reagent with which the test strip is impregnated is typically prepared from sodium tungstate in concentrations between 5 and 50 g/l, sodium molybdate in concentrations between 1 and 25 g/l and lithium sulfate in concentrations between 5 and 50 g/l.

In addition to the phosphoric acid, at least one further acid, particularly preferably hydrochloric acid, is preferably added to the reagent.

The test strip is preferably impregnated with a conventional Folin and Ciocalteau reagent solution diluted 1:1 to 1:10 with water. Solutions of this type are commercially available. A suitable preparation procedure is given in (V. L. Singleton et al., Methods in Enzymology, 1999, 299, 152-178) on page 155.

The Folin and Ciocalteau reagents are typically prepared in accordance with the following general scheme:
a) mixing of sodium tungstate and sodium molybdate in water
b) addition of o-phosphoric acid and hydrochloric acid
c) boiling of the mixture under reflux for a number of hours
d) addition of lithium sulfate.

Should a blue coloration already form during preparation of the reagent solution, this can be eliminated by boiling with a small amount of an oxidant, such as bromine.

The Folin and Ciocalteau reagent is then optionally diluted with water for impregnation of the test strip. Furthermore, further substances, such as stabilisers or buffers, can be added to the impregnation solution.

The present invention also relates to a method for the determination of the total content of phenolic components in must and wine, characterised by the following method steps:
a) provision of an aqueous sample and a test strip according to the invention
b) alkalisation of the sample by addition of a base
c) brief dipping of the test strip into the sample
d) visual and/or spectroscopic, preferably reflectometric, evaluation of the blue coloration of the test strip.

In a preferred embodiment, the alkalisation in step b) is carried out using concentrated sodium hydroxide solution.

The evaluation in step d) is preferably carried out reflectometrically with reference to a calibration curve recorded in advance.

The present invention also relates to a kit for the determination of the total content of phenolic components in must and wine, at least comprising one or more test strips according to the invention and a base for alkalisation of the sample.

The measurement range of the means and method according to the invention is between 15 and 300 mg/l.

It has thus been possible to provide a simple, rapid and reliable means and method for the determination of the content of polyphenols in musts and wines which can also be carried out on site by vintners. The essential advantages of the dry-chemical method according to the invention, besides simple handling, also include straightforward disposal owing to the small amounts of reagent.

Even without further comments, it is assumed that a person skilled in the art will be able to utilise the above description in the broadest scope. The preferred embodiments and examples should therefore merely be regarded as descriptive disclosure which is absolutely not limiting in any way.

The complete disclosure content of all applications, patents and publications mentioned above and below, in particular the corresponding application DE 102004040343.0, filed on 20 Aug. 2004, is incorporated into this application by way of reference.

### EXAMPLES

### Example 1

### Determination of Polyphenols in Grape Must/Wine—Reflectometric Evaluation of the Reaction Colour:

### Preparation of the Impregnation Solution:

Folin-Ciocalteau's phenol reagent (Merck, Art. 1.09001) is diluted in the ratio 1:5 with deionised water, and 500 mg of Marlox® (alkylpolyalkylene glycol ether, Hüls) are added per litre of solution.

### Production of the Test Sticks:

The above impregnation solution is applied to a filter paper (for example Binzer, 1450 CV; acid-washed) with exclusion of light and then dried using warm air. The paper is heat-sealed onto a white support film using hot-melt adhesive (for example Dynapol S 1272 adhesive) and cut into strips in a suitable manner to give a reaction zone measuring about 6 mm×8 mm.

### Analysis:

| | | |
|---|---|---|
| 1) Prepared sample | 5 ml | Introduce into suitable test vessel |
| solution | | |
| 2) 32% NaOH | 5 drops | Add and swirl around |

Dip the test strips into the sample to be analysed for about 2 seconds and tip excess drops off at the side.

Depending on the content of phenolic components in the sample solution, a blue coloration forms on the test zone and can be evaluated by comparison with a colour card or quantitatively using a reflectometer.

For quantitative evaluation, the test strips, after a suitable reaction time, are evaluated in a small hand diode-based reflectometer (RQflex reflectometer). The correlation between the measured relative remission (%) and the content of polyphenols is shown by Table 1.

The reference substance employed for calibration of the method was catechin hydrate in accordance with the photometric determination method. Comparative results are also obtained on use of gallic acid.

TABLE 1
Reaction time used 5 min

| Polyphenol as | |
|---|---|
| catechin hydrate | |
| (mg/l) | % rem |
| 0 | 70 |
| 25 | 55 |
| 50 | 42 |
| 75 | 33 |
| 100 | 26 |
| 150 | 20 |
| 200 | 16 |

The calibration curve is shown in FIG. 1. The amount of polyphenol (here: catechin hydrate) in mg/l is plotted on the abscissa. The remission in % is plotted on the ordinate.

### Example 2: Practical Test

Various musts/wines were investigated using the method according to the invention and using the calibration curve for reflectometric evaluation obtained under Example 1, and the results were compared with the photometric laboratory method.

White wines: the samples were diluted 1:5 with deionised water before the determination.

| Sample No. | Test strip reflectometry | Photometry |
|---|---|---|
| 1 | 260 | 249 |
| 2 | 391 | 415 |
| 3 | 385 | 361 |
| 4 | 261 | 230 |
| 5 | 259 | 270 |
| 6 | 163 | 179 |
| 7 | 421 | 382 |
| 8 | 190 | 240 |

Red wines: the samples were diluted at least 1:10 with deionised water before the determination.

| Sample No. | Test strip reflectometry | Photometry |
|---|---|---|
| 1 | 1150 | 1127 |
| 2 | 1310 | 1153 |
| 3 | 841 | 792 |
| 4 | 841 | 793 |
| 5 | 657 | 633 |
| 6 | 1002 | 850 |
| 7 | 1619 | 1510 |
| 8 | 2328 | 2467 |

1. Test strip for the determination of the total content of phenolic components in must and wine, characterised in that the test strip is impregnated with a reagent obtained by mixing and heating at least sodium tungstate, sodium molybdate and o-phosphoric acid.

2. Test strip according to claim 1, characterised in that the reagent with which the test strip is impregnated additionally comprises lithium sulfate.

3. Test strip according to claim 1, characterised in that the reagent with which the test strip is impregnated additionally comprises hydrochloric acid.

4. Test strip according to claim 1, characterised in that the reagent with which the test strip is impregnated is prepared using sodium tungstate in concentrations between 5 and 50 g/l, sodium molybdate in concentrations between 1 and 25 g/l and lithium sulfate in concentrations between 5 and 50 g/l.

5. Test strip according to claim 1, characterised in that the test strip is impregnated with a reagent which has been prepared by
mixing sodium tungstate and sodium molybdate in water
adding o-phosphoric acid and hydrochloric acid
boiling the mixture under reflux for a number of hours
adding lithium sulfate.

6. Method for the determination of the total content of phenolic components in must and wine, characterised by the following method steps:
provision of an aqueous sample and a test strip according to the invention
alkalisation of the sample by addition of a base
brief dipping of the test strip into the sample
visual and/or spectroscopic evaluation of the test strip.

7. Method according to claim 6, characterised in that the alkalisation in step b) is carried out using concentrated sodium hydroxide solution.

8. Kit for the determination of the total content of phenolic components in must and wine, at least comprising one or more test strips according to claim 1 and a base for alkalisation of the sample.
